(19) United States
(12) Patent Application Publication
(10) Pub. No.: US 2005/0069595 A1
(43) Pub. Date: Mar. 31, 2005

(54) Nitric oxide in treatment of inflammation

(75) Inventors: Luni Chen, Uppsala (SE); Jiping Da, Uppsala (SE); Goran Hedenstierna, Djursholm (SE)

Correspondence Address: BROWDY AND NEIMARK, P.L.L.C., 624 Ninth Street, N. W., Washington, DC, 20001, US

(73) Assignee: AGA AB, Lidingo (SE)

(21) Appl. No.: 10/444,074

(22) Filed: May 23, 2003

(30) Foreign Application Priority Data
Apr. 3, 2003 (SE) 0300971-9

(51) Int. Cl. A61K 33/00; A61K 31/21; A61K 31/573

(52) U.S. Cl. 424/718; 514/171; 514/509

(57) ABSTRACT

A method of treating infectious inflammation in a mammal, including man, which comprises administering to a mammal in need of such treatment, nitric oxide, in the form of gaseous nitric oxide or a nitric oxide donor, in combination with a glucocorticoid, said combination being used in a therapeutically effective amount to accomplish treatment of said inflammation. A pharmaceutical composition for treatment of such an inflammation.

### TECHNICAL FIELD OF THE INVENTION

The present invention relates to the use of nitric oxide, in the form of gaseous nitric oxide or a nitric oxide donor, in combination with a glucocorticoid for the manufacture of a medicament for treating infectious inflammation in a mammal, including man, to a method for treating such an inflammation, and to a pharmaceutical composition for treatment of such an inflammation.

2. Background Art

Numerous experimental and clinical studies demonstrate improved oxygenation of blood and relief or attenuation of pulmonary hypertension by inhaled nitric oxide (inhaled NO, INO) in the treatment of acute lung injury. These effects are brought about by selective dilation of pulmonary vessels in ventilated lung parenchyma. INO has also an anti-inflammatory effect and inhibits the expression of many genes thought to be involved in inflammatory diseases. These include chemokines, adhesion molecules, tumor necrosis factor alpha (TNF-α), interleakins, nuclear factor kappa B (NF-κB) and cyclooxygenase-2 (COX2). However, the understanding of the interactions between NO and inflammatory markers is far from complete. Increased knowledge may open new routes to suppress inflammation. INO may exert extra-pulmonary effects, e.g. prevention of clotting, improved urine output, but no real evidence of systemic anti-inflammatory effects has been shown. (Kang, J. L. et al., J. Appl. Physiol. (2002) 92(2), 795-801; Kinsella, J. P. et al., Pediatr. Res. (1997) 41(4), 457-463; Troncy, E. et al., Br. J. Anaesth. (1997) 79(5), 631-640; Ballevre, L. et al., Biol. Neonate. (1996) 69(6), 389-398; Wraight, W. M. et al., British Journal of Anaesthesia (2001) 86(2), 267-269.)

The main therapeutic action of inhaled NO is pulmonary vasodilation. Two conceptions have made this application important: First, that the action of NO is limited to the pulmonary circulation. Second, that since NO is administered in inspired air it acts preferentially on ventilated alveoli. (Rang, H. P. et al., Pharmacology (1995), Churchill Livingstone.) Thus, prior therapeutic use of NO has been related to local, pulmonary, effects.

Glucocorticoids (GCs) are steroid hormones produced by the adrenal glands by stimulation of the hypothalamus-pituitary-adrenal (HPA) axis. It is generally accepted that their potent anti-inflammatory and immuno-modulatory actions are due to inhibition of the activity of transcription factors, such as activator protein-1 (AP-1) and NF-κE, that are involved in modulation of pro-inflamatory genes. The effects of GCs are exerted through the glucocorticoid receptor (GR), a ligand-induced transcription factor, which belongs to the nuclear receptor superfamily. GR controls transcription by two major modes of action. One involves binding of GR homodimers to glucocorticoid response elements (GREs) in regulatory sequences of GR target genes. Another mode of action is that GR modulates the activity of other transcription factors such as AP-1, NF-KB and Stat5, independently of direct DNA contact, a process designated as cross talk. The GR itself does not need to bind to DNA for this second mode of action. (Adcock, I. M. et al., Immunology and Cell Biology (2001) 79(4), 376-384; De Bosscher, K. et al., J. Neuroimmunol. (2000) 109(1), 16-22; Refojo, D. et al., Immunology and Cell Biology (2001) 79(4), 385-394; Reichardt, H. M. et al., The EMBO Journal (2001) 20(24), 7168-7173.)

The clinical use of glucocorticoids comprises replacement therapy for patients with adrenal failure, anti-inflammatory/immunosuppressive therapy, and use in neoplastic disease. Generally, glucocorticoids have been considered to possess anti-inflammatory and immunosuppressive activities. They inhibit both the early and the late manifestations of inflammation. Thus, glucocorticoids are used for anti-inflammatory/immunosuppressive therapy in asthma, in inflammatory conditions of skin, eye, ear or nose (e.g. eczema, allergic conjunctivitis or rhinitis), in hypersensitive states, miscellaneous diseases with autoimmune and inflammatory components, and to prevent graft-versus-host disease. (Rang, H. P. et al., Pharmacology (1995), Churchill Livingstone.) However, the above-mentioned uses of glucocorticoids are all related to non-infectious inflammations. Traditionally, antibiotic therapy has been preferred for infectious diseases.

Bacterial pneumonia is caused by a pathogenic infection of the lungs. Examples of infectious agents are pneumococcal agents; Haemophilus influenzae; Klebsiella, Staphylococcus, and Legionella species; gram-negative organisms; and aspirated materials. Bacteria from the upper airways or, less commonly, from hematogenous spread, find their way to the lung parenchyma. Once there, a combination of factors (including virulence of the infecting organism, status of the local defences, and overall health of the patient) may lead to bacterial pneumonia. (Stephen, J. (2003), Bacterial Pneumonia, <http://www.emedicine.com/emerg/topic465.htm>)

The mainstay of drug therapy for bacterial pneumonia is antibiotic treatment. The role of glucocorticoids in acute bacterial pneumonia is not yet clear. Classic teaching warns that the use of glucocorticoids in infection may impair the immune response. However, recent findings show that local pulmonary inflammation may be reduced with systemic glucocorticoids. (Toshinobu Yokoyama et al., J. Infect. Chemother. (2002) 8(3), 247 -251; Lefering R. et al., Crit. Care Med. (1995) 23, 1294-1303.)

Sepsis or septic shock is systemic inflammatory response secondary to a microbial infection. Prior to the introduction of antibiotics in clinical practice, gram-positive bacteria were the principal organisms causing sepsis. More recently, gram-negative bacteria have become the key pathogens causing severe sepsis and septic shock.

The treatment of patients with septic shock consists of the following 3 major goals: (1) Resuscitate the patient from septic shock using supportive measures to correct hypoxia, hypotension, and impaired tissue oxygenation. (2) Identify the source of infection and treat with antimicrobial therapy, surgery, or both. (3) Maintain adequate organ system function guided by cardiovascular monitoring and interrupt the pathogenesis of multiorgan system dysfunction.

While theoretical and experimental animal evidence exists for the use of large doses of corticosteroids in those with severe sepsis and septic shock, all randomized human studies (except one from 1976) found that corticosteroids did not prevent the development of shock, reverse the shock state, or improve the 14-day mortality rate. Therefore, no support exists in the medical literature for the routine use of high doses of corticosteroids in patients with sepsis or septic shock (see further below). (Sharma, S., Mink, S. (2003), Septic Shock, <http://www.emedicine.com/med/topic2101.htm>)

However, a recent trial demonstrated positive results of stress-dose administration of corticosteroids in patients with severe and refractory shock (Briegel, J. et al., Crit. Care Med. (1999) 27, 723-732).

Acute respiratory distress syndrome (ARDS) is defined as an acute condition characterized by bilateral pulmonary infiltrates and severe hypoxemia in the absence of evidence for cardiogenic pulmonary edema. ARDS is associated with diffuse damage to the alveoli and lung capillary endothelium. Risk factors for ARDS include direct lung injury, systemic illnesses, and injuries. The most common risk factor for ARDS is sepsis. Other nonthoracic conditions contributing to the risk for developing ARDS include trauma with or without massive transfusion, acute pancreatitis, drug overdose, and long bone fracture. The most common direct lung injury associated with ARDS is aspiration of gastric contents. Other risk factors include various viral and bacterial pneumonias, near drowning, and toxic inhalations. (Hardman, E. M., Walia, R. (2003), Acute Respiratory Distress Syndrome, <http://www.emedicine.com/med/topic70.htm>)

No drug has proved beneficial in the prevention or. management of ARDS. The early administration of corticosteroids in septic patients does not prevent the development of ARDS. Inhaled nitric oxide (NO), a potent pulmonary vasodilator seemed promising in early trials but, in larger controlled trials, did not change mortality rates in adults with ARDS. A potential role for corticosteroids may exist in patients with late ARDS (fibroproliferative phase) because they decrease inflammation by suppressing migration of polymorpho-nuclear leukocytes and reversing increased capillary permeability. This may be considered rescue therapy in selected patients, but widespread use is not recommended pending the results of an ARDS Network trial now underway, (Kang, J. L. et al., J. Appl. Physiol. (2002) 92(2), 795-801; Reichardt, H. M. et al., The EMBO Journal (2001) 20(24), 7168-7173.)

As is understood from the preceding review of several infectious conditions, the use of glucocorticoid therapy in patients with infectious inflammations, such as sepsis and septic shock, is controversial and much debated. Large randomized studies and meta-analyses have failed to show a mortality benefit and have even indicated that steroid therapy may be harmful (Cronin, L. et al., Crit. Care Med. (1995) 23, 1430-1439; Lefering, R. et al., Crit. Care Med. (1995) 23, 1294-1303). A therapy directed at the microbial cause of the disease, such as the use of antibiotics, is generally preferred. Further, when glucocorticoid therapy has been suggested for septic patients, it is aimed at patients with adrenal function abnormalities (Annane, D., et al., JAMA (2002) 288, 862-871). Glucocorticoid therapy is thus not an obvious and general choice for a physician facing a patient suffering from sepsis or septic chock.

To sum up, bacterial pneumonia, septic shock and ARDS are, as discussed above, examples of infectious inflammations. Therapy of such inflammations has traditionally been focused on the underlying infection, i.e. different kinds of antibiotics have been used. In some cases glucocorticoid therapy has been suggested, primarily in connection with specific additional conditions. It has, however, also been advised against the glucocorticoid therapy in bacterial pneumonia, septic shock and ARDS for different reasons, e.g. that no therapeutic effect has been obtained or that side effects have occurred.

WO 99/20251 (Zapol et al.) discloses methods for decreasing or preventing non-pulmonary ischemia-reperfusion injury and non-pulmonary inflammation. Examples of non-pulmonary inflammation are arthritis, myocarditis, encephalitis, transplant rejection, systemic lupus erythematosis, gout, dermatitis, inflammatory bowel disease, hepatitis, and thyroiditis. The methods include causing a mammal to inhale gaseous nitric oxide. The NO gas diminishes the ability of circulating leukocytes or platelets to become activated and contribute to an inflammatory process at the site of ischemia-reperfusion or inflammation in the non-pulmonary tissue. In combination with the inhaled NO gas, a second compound that potentiates the therapeutic effect of gaseous NO can be administered. The second compound can be, above all, a phosphodiesterase inhibitor, but also, for example, a glucocorticoid. However, this document relates to the treatment of non-infectious inflammations only. The use of i.a. glucocorticoids for the treatment of non-infectious inflammations is well known and the contribution of the glucocorticoid in the described methods is complementary to the contribution of NO rather than providing for a synergistic effect (i.e. a more enhanced effect than the sum of the individual INO and steroid effects).

Finally, although U.S. Pat. No. 5,837,698 and U.S. Pat. No. 5,985,862 (Tjoeng et al.) touch upon nitric oxide as an agent to enhance the actions of corticosteroids in the treatment of several various diseases, no examples of their collective action are given and no explicit references to infectious inflammations are made. The real disclosures of these documents are steroid nitrite and/or nitrate ester compounds, combining a steroid moiety and a nitric oxide donating moiety in one molecule.

### SUMMARY OF THE INVENTION

The present invention is based on the finding that there are complex interactions between glucocorticoids and nitric oxide in the inflammatory process. From these interactions, it has appeared that a strong synergistic effect is obtained. It is likely that nitric oxide up-regulates the expression of the glucocorticoid receptor and in combination with administration of glucocorticoids blunts the inflammatory response.

The object of the present invention is to provide a medicament for treating infectious inflammations. More specifically, the purpose of said medicament is to benefit by the above-mentioned synergistic effect.

The above-mentioned object as well as other objects of the invention, which should be apparent to a person skilled in the art after having studied the description below, are accomplished by the use of nitric oxide, in the form of gaseous nitric oxide or a nitric oxide donor, in combination with a glucocorticoid for the manufacture of a medicament for treating infectious inflammations in a mammal, including man, said combination being used in a therapeutically effective amount to accomplish treatment of said inflammation. The objects are also accomplished by a method of treating infectious inflammations and a pharmaceutical composition for treatment of infectious inflammations.

The use of nitric oxide in combination with a glucocorticoid utilizes the abovementioned synergistic effect. The effect is striking in comparison with the effect of nitric oxide or glucocorticoids alone. The effects of the combined medicament are further illustrated in the Example below.

The inflammatory response to an infectious inflammation involves down-regulation of the glucocorticoid receptor. Thus, the use of nitric oxide, which up-regulates the glucocorticoid receptor, in combination with a glucocorticoid, is particularly preferable for treating infectious inflammations.

An infectious inflammation may be caused by bacteria, fungi, viruses, mycoplasma, protozoa, helminths or insects. Bacterial infections may for example be caused by bacteria of the Bacteroides, Corynebacterium, Enterobacter, Enterococcus, Escherichia coli, Staphylococcus and Streptococcus genera or species.

Examples of infectious inflammations, suitable for treatment by use of nitric oxide and a glucocorticoid according to the invention, are sinusitis, upper respiratory infection, bronchiectasis, bronchitis and chronic bronchitis, bacterial pneumonia, urinary tract infection, meningitis, acute respiratory distress syndrome, myocarditis, pericarditis, endocarditis, rheumatic fever, sepsis, and septic arthritis.

Preferred inflammations for treatment according to the invention are sepsis, bacterial pneumonia and acute respiratory distress syndrome.

Moreover, systemic effects of the medicament are seen with good effects in the lungs, the kidney and the liver.

The nitric oxide and the glucocorticoid may be administered simultaneously.

However, more preferably the nitric oxide is administered separately from the glucocorticoid. Separate administration allows for independent control of the doses of nitric oxide and glucocorticoid, respectively. The optimal dose ratio of the two components may vary depending on the age, sex, condition etc. of the patient and the severity of the disease.

Most preferably, the nitric oxide is administered before the glucocorticoid. Early administration of nitric oxide allows for priming, i.e. up-regulation, of the glucocorticoid receptor ahead of administration of the glucocorticoid. Such sequential administration of nitric oxide and glucocorticoid provides an excellent basis for the synergistic effect of the invention. The time interval between the administration of nitric oxide and the administration of glucocorticoid may be from about 1 minute, for inflammation in e.g. the lungs, to about 30 minutes, for inflammation in more distant tissues, such as the liver or kidneys.

The nitric oxide can be administered as gaseous nitric oxide or as a nitric oxide donor. The preferred administration of gaseous nitric oxide as inhalable nitric oxide provides for fast onset and offset of its effect. It also represents a convenient route of administration to patients requiring mechanical ventilation.

The glucocorticoid can be administered by any known route of administration for steroids, such as orally, by inhalation, by intramuscular or subcutaneous injection, by intravenous infusion or injection, or by intracutaneous or intra-articular injection. A preferred route of administration is, however, intravenously, since the micro-circulation in severely sick patients, such as patients with sepsis, may be poor and thus limit the absorption of drugs.

Further, the medicament according to the present invention can be an inhalable medicament.

Inhalable nitric oxide is present in a carrier gas or a gas mixture, e.g. admixed with nitrogen to protect the nitric oxide from oxidation. The concentration of nitric oxide in such a carrier gas or gas mixture is normally within the range of 0.1-180 ppm, preferably 1-80 ppm, and more preferably 1-40 ppm.

The dose of glucocorticoid in the medicament of the present invention is normally within the range of 0.1 to 10 mg/kg body weight.

As an alternative to inhalation of gaseous nitric oxide, nitric oxide can be administered in the form of a nitric oxide donor. Examples of suitable nitric oxide donors are S-nitrosocysteine, nitroprusside, nitrosoguanidine, glycerol trinitrate, isoamyInitrite, inorganic nitrite, aside, hydroxylamine, nitroglycerin, isosorbide dinitrate and pentaerithrityl tetranitrate.

Any glucocorticoid triggering the glucocorticoid receptor is suitable for use according to the invention. Examples of preferred glucocorticoids are hydrocortisone, cortisone, corticosterone, prednisolone, prednisone, methylprednisolone, triamcinolone, dexamethasone, bethametasone, beclomethasone, budesonide, deoxycortone, fluocinoide, clobetasone and corticotrophin.

### BRIEF DESCRIPTION OF THE DRAWINGS

FIG. 1 shows the different protocols used in the Example.

FIGS. 2A and 2B shows mean pulmonary artery pressure (MPAP) results and arterial oxygenation (PaO₂) results, respectively, from the Example. The data are from healthy animals (star) and from animals exposed to endotoxine alone (triangle), endotoxin+inhaled nitric oxide (circle), endotoxin+steroid (diamond), and endotoxin+inhaled nitric oxide and steroid (plus).

FIG. 3 shows histology changes of lung tissue by endotoxin alone (LPS), endotoxin+inhaled nitric oxide (LPS+INO), endotoxin+steroid (LPS+Steroid), and endotoxin+inhaled nitric oxide and steroid (LPS+INO+Steroid), compared with lungs from healthy piglets (Healthy). Endotoxin induced acute inflammatory cell infiltrates in alveolar septa and around bronchial walls, swelling of epithelial and endothelial cells, and pulmonary edema and hemorrhage.

FIG. 4 shows histology changes of liver tissue by endotoxin alone (LPS), endotoxin+inhaled nitric oxide (LPS+INO), endotoxin+steroid (LPS+Steroid), and endotoxin+inhaled nitric oxide and steroid (LPS+INO+Steroid), compared with liver from healthy piglets (Healthy). Endotoxin induced acute inflammatory cell infiltrate in the connective tissue in the periphery of lobules, massive liver cell congestion, and necrosis, Kupffer cell reactive hyperplasia, and hemorrhage.

FIG. 5 shows histology changes of kidney tissue by endotoxin alone (LPS), endotoxin+inhaled nitric oxide (LPS+INO), endotoxin+steroid (LPS+Steroid), and endotoxin+inhlaed nitric oxide and steroid (LPS+INO+Steroid), compared with kidney from healthy piglets (Healthy). Endotoxin induced acute inflammatory cell infiltration, edema, and the destruction of glomerular structure, and necrosis of cells in glomeruli.

FIG. 6 shows expression of glucocorticoid receptor in lung tissue in piglets exposed to endotoxin alone (LPS) or endotoxin+inhaled nitric oxide (LPS+INO).

FIG. 7 shows expression of NF-KB in lung tissue in piglets exposed to endotoxin alone (LPS) or endotoxin plus inhaled nitric oxide (LPS+INO).

### EXAMPLE

Materials and Methods

Animal Preparation

The study was approved by the Animal Research Ethics Committee of Uppsala University. Thirty-eight piglets of Swedish country breed, weighing 22-28 kg, were used. Anesthesia was induced with atropine i.m., 0.04 mg/kg, tiletamine/zolazepam (Zoletid, Virbac Laboratories), 6 mg/kg, and xylazize chloride (Rompun, Bayer AG, Germany), 2.2 mg/kg, and maintained with a continuous infusion of a hypnotic, clormethiazole (Heminevrin, Astra, Södertälje, Sweden), 400 mg/h, pancuronium, 2 mg/h, and fentanyl, 120 mg/h. Pre-warmed (38° C.) isotonic saline 500 ml/h was given i.v. to prevent dehydration. The animals were placed in the supine position for the remainder of the study.

After induction of anesthesia a tracheotomy was performed and a cuffed tracheal tube was inserted. Mechanical ventilation was provided in volume-controlled mode (Servo 900 C, Siemens-Elema, Lund, Sweden) at a respiratory frequency of 22±2 breaths per minute (mean±SD), an inspiratory to expiratory ratio of 1:2, and an end-inspiratory pause of 5% of the respiratory cycle. The minute ventilation was adjusted to obtain an end-tidal CO₂. tension (PetCO₂) of 33-45 mmHg (4.4-6.0 kPa) in the initial control situation, and was then kept constant throughout the experiment. The mean tidal volume was 10±1.4 ml/kg. A positive end-expiratory pressure (PEEP) of 5 cm H₂O was applied. The inspired fraction of oxygen (FIO₂) was 0.5. A triple-lumen balloon-tipped catheter (Swan Ganz no. 7F) was introduced into the pulmonary artery for blood sampling and pressure recording. The contralateral jugular vein and right carotid artery were also catheterized for pressures recording, blood sampling and infusion. Mean arterial pressure (MAP), mean pulmonary arterial pressure (MPAP), heart rate (HR), central venous pressure (CVP), pulmonary capillary wedge pressure (PCWP), and cardiac output (Qt) were recorded. Douglas and bladder catheters were inserted for measurement of urine flow and ascitic fluid.

Mixed venous and arterial blood samples were collected for blood gas analysis (ABL 500, Radiometer, Copenhagen, Denmark) and determination of oxygen saturation and hemoglobin concentration (OSM 3, Radiometer, Copenhagen, Denmark). Hemoximeter data were corrected for pig blood.

Protocol

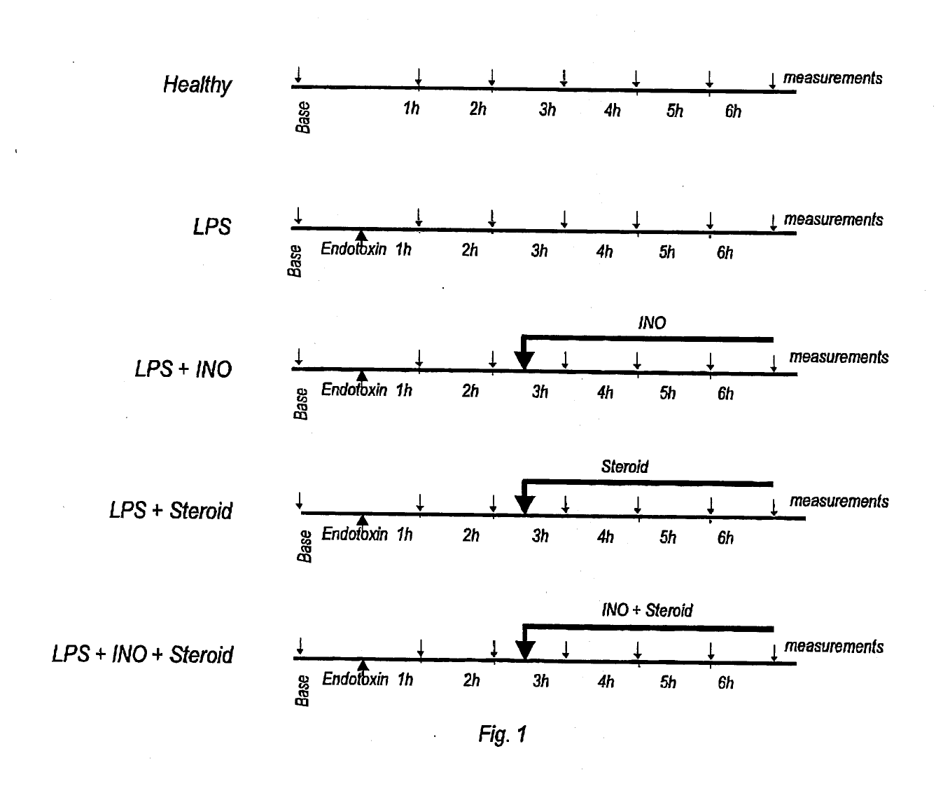

The pigs were divided into 5 groups:
1. Normal control group
2. Endotoxin (lipopolysaccharide, LPS) group
3. LPS+INO group
4. LPS+steoid group
5. LPS+INO+steroid group
   The different protocols are shown schematically in FIG. 1 and are described below.
   1. Healthy Control Group (n=8)

Anesthesia, surgery preparation and catheterizations were followed by 30 min of rest. Baseline measurements were then made. The pigs were followed for another 6 hours to establish control data over the whole study period. The pigs were killed after the last measurement with an intra-venous injection of KCl; and tissue samples were taken from the lung, liver and kidney for morphological and biochemical studies.

2. LPS group (n=8)

Anesthesia, surgery and catheterizations were similar to the controls. After baseline measurements, acute lung injury and septic shock was induced by intravenous infusion of LPS 25 μg/kg/h in saline for 2.5 h, followed by 10 μg/kg/h for the remainder of the study. The pigs were followed by hemodynamics and blood gas measurements and were killed at the end of the experiment and tissue samples were taken, as described above.

3. LPS+INO group (n=8)

LPS infusion was started after baseline measurements and continued for 6 hours. Two and half hours after commencement of the endotoxin infusion, inhalation of NO 30 ppm was started and maintained for 3.5 hours. Measurements were taken every hour to check for any protective effect of INO therapy on the lung and extra-pulmonary organs.

4. LPS+Steroid (S) group (n=7)

The protocol was the same as above, but the pigs received a steroid, hydrocortisone i.v. (Solucortef®, Pharmacia), 3.5 mg/kg, instead of INO 2.5 hours after the start of endotoxin infusion.

5. LPS+INO+S group (n=7)

This group received endotoxin over 6 hours, as described above, and 2.5 hours after the start of endotoxin administration steroids were given i.v. and INO, 30 ppm, was commenced and continued for the remaining 3.5 hours study period.

NO Administration

NO, 1000 ppm in N₂, was added to a mixture of O₂/N₂ and administered through the low-flow inlet of the ventilator. The inspired gas was passed through a canister containing soda lime to absorb any NO₂. The inhaled NO was set to 30 ppm and the concentration of inspired NO₂ was always less than 0.2 ppm. The concentrations of inspired NO and NO₂ were measured continuously, by chemiluminescence (9841 NOₓ, Lear Siegler Measurement Controls Corporation, Englewood, CO, USA), in the inspiratory limb of the ventilator tubing. FIO₂ was checked after addition of NO and kept stable at the pre-INO level.

Immunohistochomistry

Immunohistochemical detection of GR and NF-κB was achieved with standard streptavidin-biotin-peroxidase detection techniques (GR: Santa Cruz Biotechnology, Inc Catalogue No. sc-1004 USA, Rabbit Polyclonal Antibody, dilution 1:200; NF-XB: SIGMA, Product No. N5823 Germany, dilution 1:100), Pilot experiments showed that autoclave or microwave antigen retrieval and overnight incubation of the primary antibodies yielded the best sensitivity. The antibodies were detected with the peroxidase-anti-peroxidase method using 3-amino-9-ethyl-carbazole (AEC, SIGMA Catalogue No. A-6926 Germany) as chromogen. All slides were counterstained with 0.1% Certified Haematoxylin (SIGMA Catalogue No. MHS-16 Germany).

Image Analysis of Immunohistochemistry

An image-analysis system consisting of a 12-bit cooled charge-coupled device camera (Sensys KAF 1400, Photometrics, Tucson, Ariz.) mounted on a fully automated Leica (Wetzlar, Germany) DM RXA microscope was used to digitize grey-scale images to a dual-Pentium 200 MHz host computer. Microscope settings were kept constant throughout all measurements (×40 objective, Leica PL Fluotar 40×/0.75). A stabilized 12 V tungsten-halogen lamp (100 W) was used for illumination. Microdensitometry was performed with a custom-designed filter manufactured by Omega Optical (Brattleboro, Vt.) for absorbency measurement of the Vector red substrate (central wavelength 525 nm, half bandwidth 10±2 nm). The optimal central wavelength was determined by measurement of the substrate in a Leica MPV SP microscope photometer system (courtesy of Leica).

Statistical Analysis

The values are expressed as the mean±SD. Significant differences were evaluated by two-way analysis of variance followed by Student-Newman-Keuls test. Statistical significance was assumed at P<0.05.

Results

Haemodynamics and Arterial Oxygenation

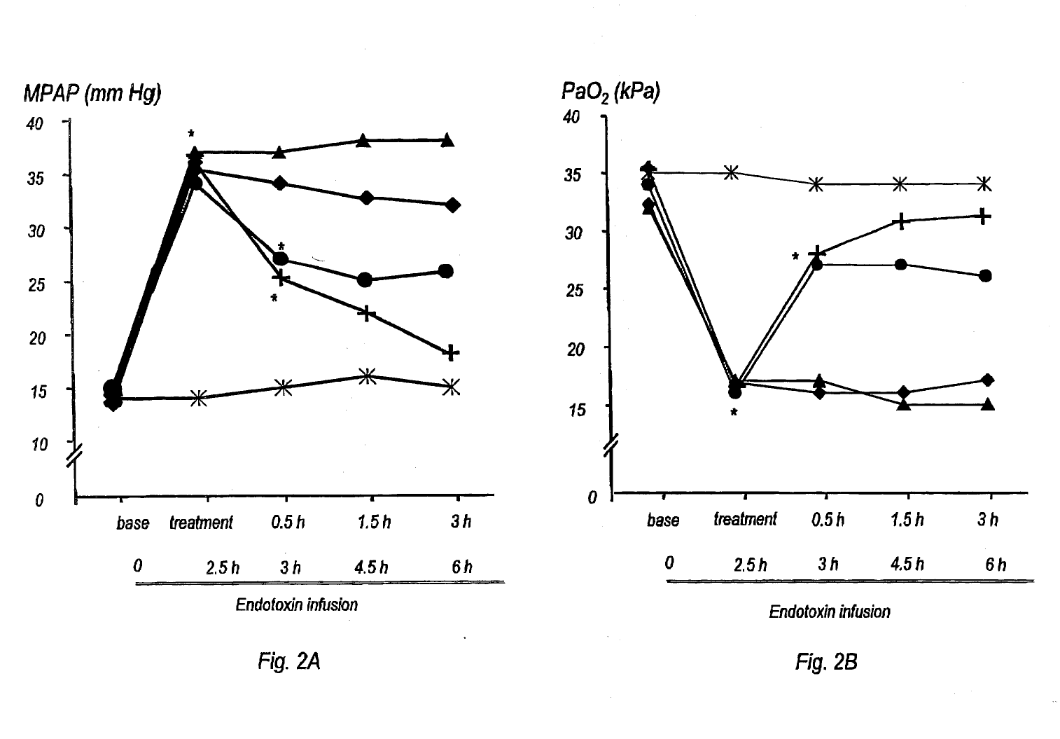

There were no significant differences in baseline hemodynamics and arterial oxygenation among the five study groups. In the endotoxin group, LPS infusion caused an increase in pulmonary artery pressure that remained elevated at twice or three times the baseline level (FIG. 2). PaO₂ was significantly reduced half an hour after onset of the LPS infusion and it continued to decrease for a few hours and then remained at less than half the baseline level (FIG. 2). INO attenuated the increase in MPAP, but it was significantly higher, by 75-80%, than in healthy controls (p<0.01) (FIG. 2). INO prevented part of the fall in PaO₂. Pigs that received steroids only, during endotoxin infusion, had as high MPAP as the pigs that received endotoxin alone. Thus steroids showed no clear effect on. Also, PaO₂ did not improve by steroid treatment but remained as low as in the endotoxin group.

Finally, pigs that received both INO and steroids showed a successive fall in MPAP that was no longer significantly different from healthy controls at the end of the experiment after 6 hours of endotoxin infusion (FIG. 2). Moreover, PaO₂ improved during the therapy and was no longer significantly different from the healthy controls at the end of the experiment.

Histological Changes

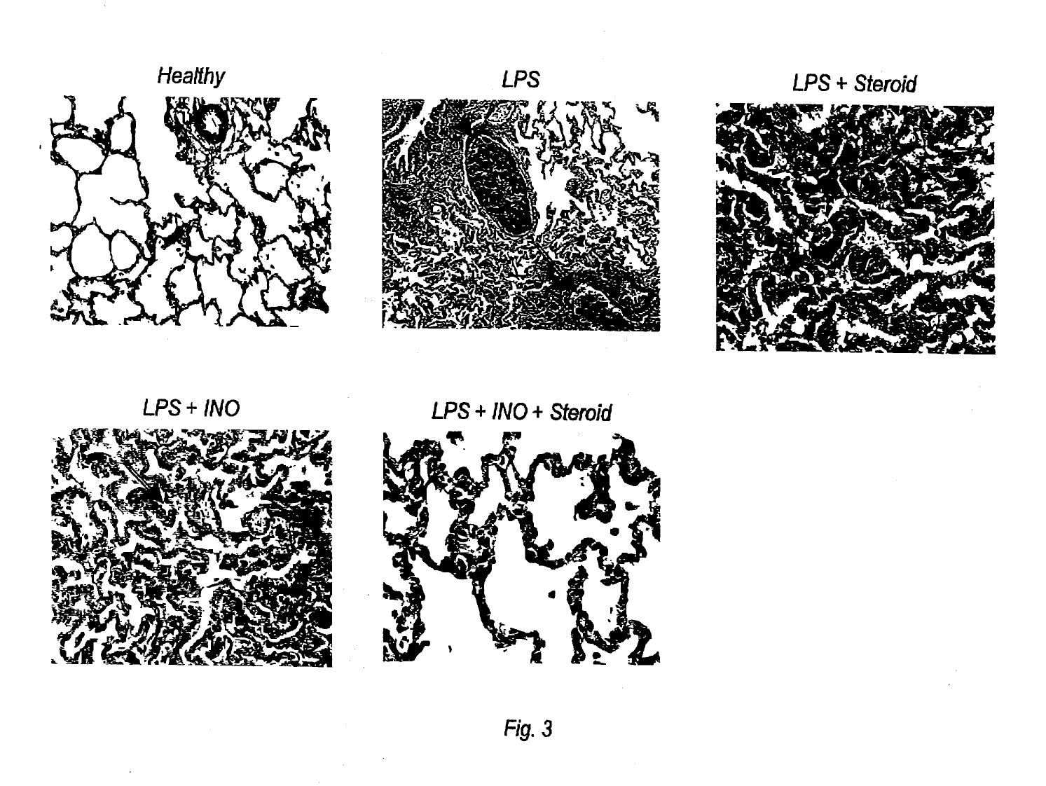

See Table 1 and FIGS. 3, 4 and 5.

FIG. 3 shows histology changes of lung tissue. Endotoxin induced acute inflammatory cell infiltrates in alveolar septa and around bronchial walls, swelling of epithelial and endothelial cells, and pulmonary edema and hemorrhage. Steroid treatment (LPS+Steroid) restored or prevented some of the damage. The lungs in the LPS+INO group had even less of these changes and the lungs exposed to LPS+INO+Steroid had only minor changes, and had thus a histology close to normal. However, some inflammatory cell infiltration and thicker alveolar septa and some edema were still seen. Arrows indicate changes as described above.

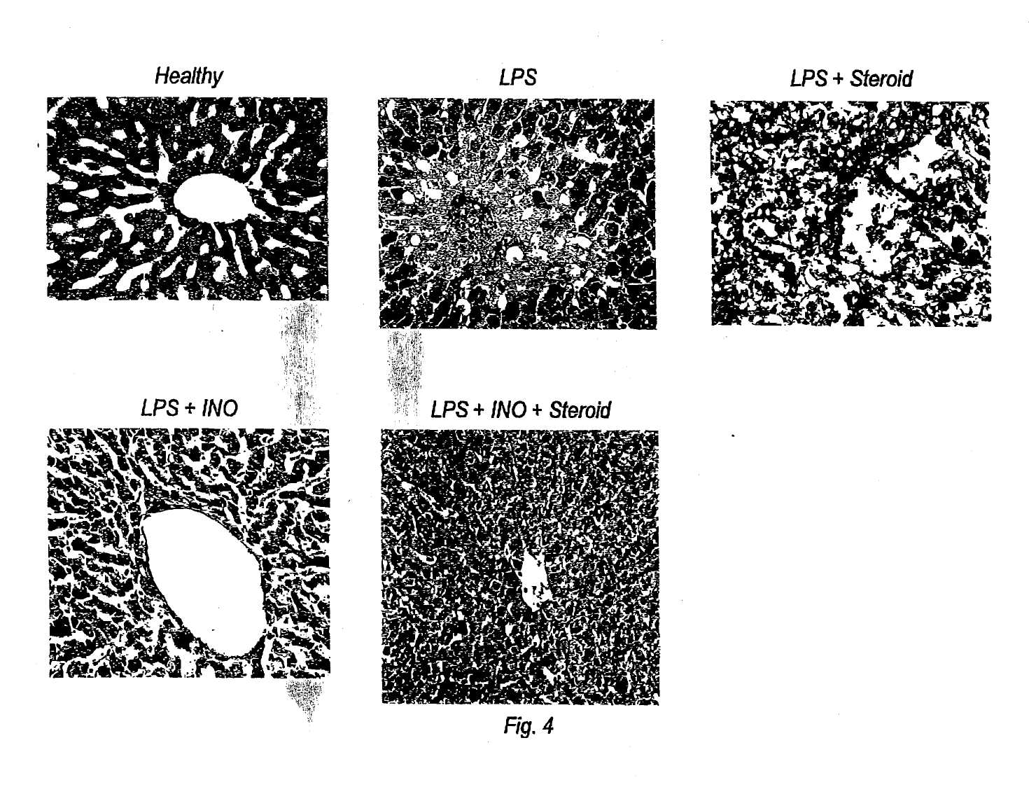

FIG. 4 shows histology changes of liver tissue. Endotoxin induced acute inflammatory cell infiltrate in the connective tissue in the periphery of lobules, massive liver cell congestion, and necrosis, Kupffer cell reactive hyperplasia, and hemorrhage. In pigs exposed to endotoxin plus steroid (LPS+Steroid), hepatic degeneration was a little less than that in the LPS group. In the liver exposed to endotoxin plus inhaled nitric oxide (LPS+INO), these changes were much less than in the LPS group. In the group exposed to endotoxin plus both inhaled nitric oxide and steroid (LPS+INO+Steroid group), structure of hepatic tissue was close to the liver from healthy controls, Thus, there was almost no necrosis, although increased number of Kupffer cells and some inflammatory cell infiltration were seen. Arrows indicate changes as described above.

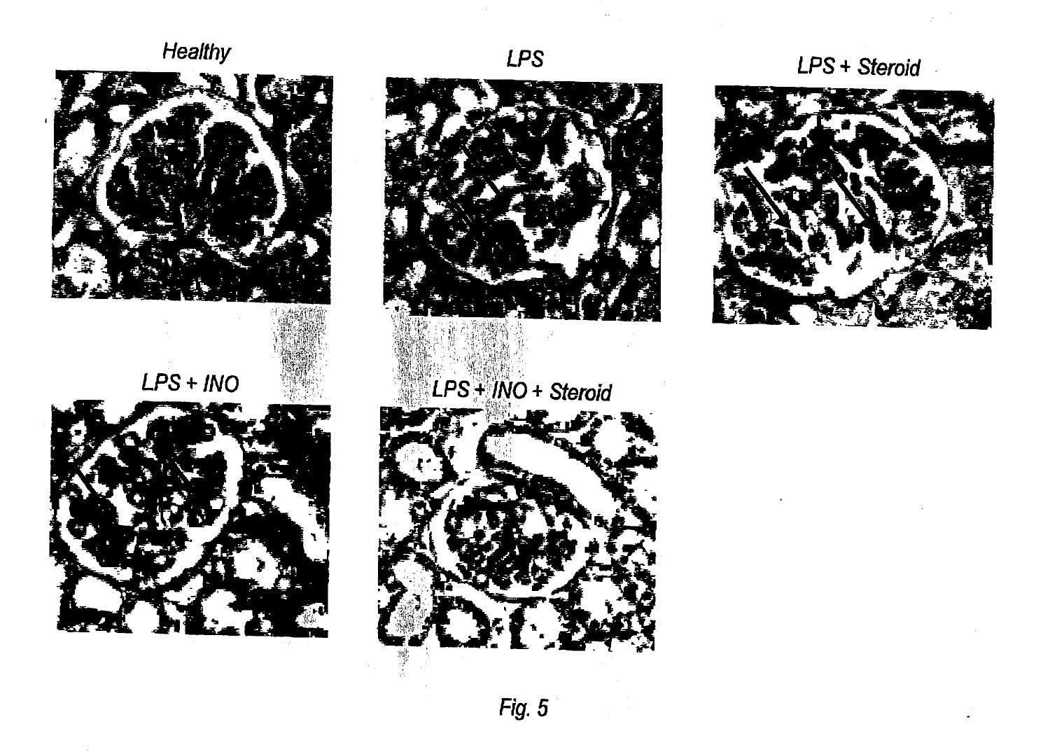

FIG. 5 shows histology changes of kidney tissue. Endotoxin induced acute inflammatory cell infiltration, edema, and the destruction of glomerular structures and necrosis of cells in glomeruli. In the kidney exposed to endotoxin plus steroid (LPS+Steroid), these changes were a little less marked than in the LPS group. Thus, there were more cells in the glomeruli (indicating less degeneration). In the pigs exposed to endotoxin plus inhaled nitric oxide (LPS+INO), the changes were even less marked than in the LPS+Steroid group. In the group exposed to endotoxin plus both inhaled nitric oxide and steroid (LPS+INO+Steroid), the glomerular structure was maintained. However, inflammatory cell infiltration, swelling of glomeruli and decrease in Bowman's capsule space were still observed. Arrows indicate changes as described above.

Table 1 shows histological changes of lung (upper panel), liver (middle panel) and kidney (lower panel) by endotoxin alone (LPS), endotoxin+inhaled nitric oxide (LPS+INO), endotoxin+steroid (LPS+S), and endotoxin+inhaled nitric oxide and steroid (LPS+INO+S), compared with healthy pigs (Healthy). +indicates severity of changes on a five degree scale (−, +, ++, +++, ++++). * means p<0.05 versus LPS. All types of changes were also significantly different from healthy controls except for LPS+INO+S pigs that showed no or minor pulmonary edema or hemorrhage, no destruction or necrosis of lobular structure in the liver, and no necrosis in the kidney.

TABLE 1
Histological changes of lung, liver and kidney
tissues

| | | | | | |
|---|---|---|---|---|---|
| Changes | Healthy | LPS | LPS + INO | LPS + S | LPS + INO + S |

Lung

| | | | | | |
|---|---|---|---|---|---|
| Inflam- | − | ++++ | +++ | ++++ | ++/* |
| matory cell | | | | | |
| infiltrate | | | | | |
| Congestion | − | ++++ | +++ | +++ | ++/* |
| Pulmonary | − | ++++ | ++(+)/* | +++ | +/* |
| edema | | | | | |
| Hemorrhage | − | ++++ | +/* | ++(+)/* | +(−)/* |

Liver

| | | | | | |
|---|---|---|---|---|---|
| Inflam- | − | ++++ | +++ | ++++ | ++/* |
| matory cell | | | | | |
| infiltrate | | | | | |
| Congestion | − | ++++ | ++ | +++ | ++/* |
| and | | | | | |
| hemorrhage | | | | | |
| Necrosis | − | ++++ | +(+)/* | +++ | +/−/* |
| Destruction | − | ++++ | + | +++ | −/* |
| of the | | | | | |
| lobular | | | | | |
| structure | | | | | |

Kidney

| | | | | | |
|---|---|---|---|---|---|
| Inflam- | + | ++++ | +++ | ++++ | ++/* |
| matory | | | | | |
| cell infiltrate | | | | | |
| Edema/ | − | ++++ | +++ | +++ | +(+)/* |
| Hemorrage | | | | | |
| Destruction | − | ++++ | ++(+)/* | +++ | +(+)/* |
| of | | | | | |
| glomerular | | | | | |
| structure | | | | | |
| Necrosis | − | ++++ | ++/* | +++ | +/−/* |

Immunohistochemistry
Glucocorticoid Receptor, GR

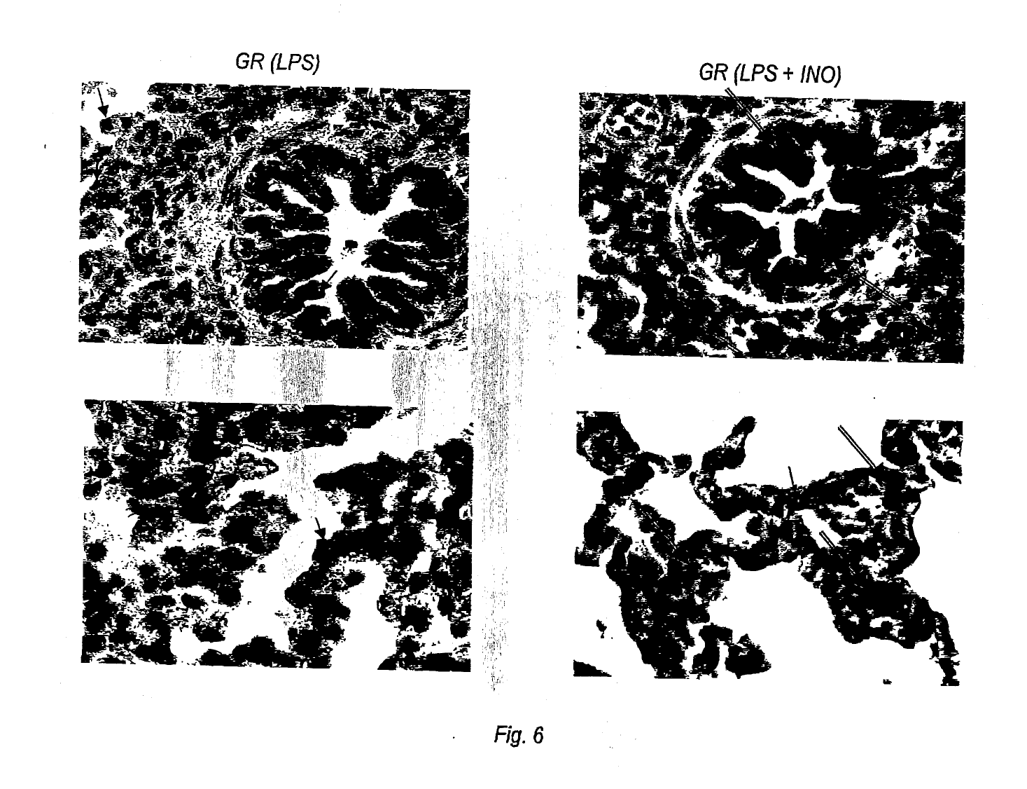

See Table 2 and FIG. 6.

FIG. 6 shows expression of glucocorticoid receptor in lung tissue. In the LPS group, weak expression of GR in few cells was observed, less than in normal tissue (not shown here). The expression of GR was significantly raised in the LPS+INO group, with a large number of cells with increased intensity of staining (dark color, indicating GR expression); The staining was seen in inflammatory cells and epithelial cells of airways and alveoli. Arrows indicate positively stained cells.

Table 2 shows expression of glucocorticoid receptor in lung and liver tissue in piglets exposed to endotoxin alone (LPS) or endotoxin+inhaled nitric oxide (LPS+INO), compared with lungs from healthy piglets (Healthy).+indicates amount of expression. * means p<0.05 versus healthy control. § means p<0.05 versus LPS.

TABLE 2
GR expression (results of immunohistochemical
staining)

| Tissue | Healthy | LPS | LPS + INO |
|---|---|---|---|
| Lung | +(+) | +(−)* | ++++*§ |
| Liver | + | −−* | ++*§ |

NF-κB

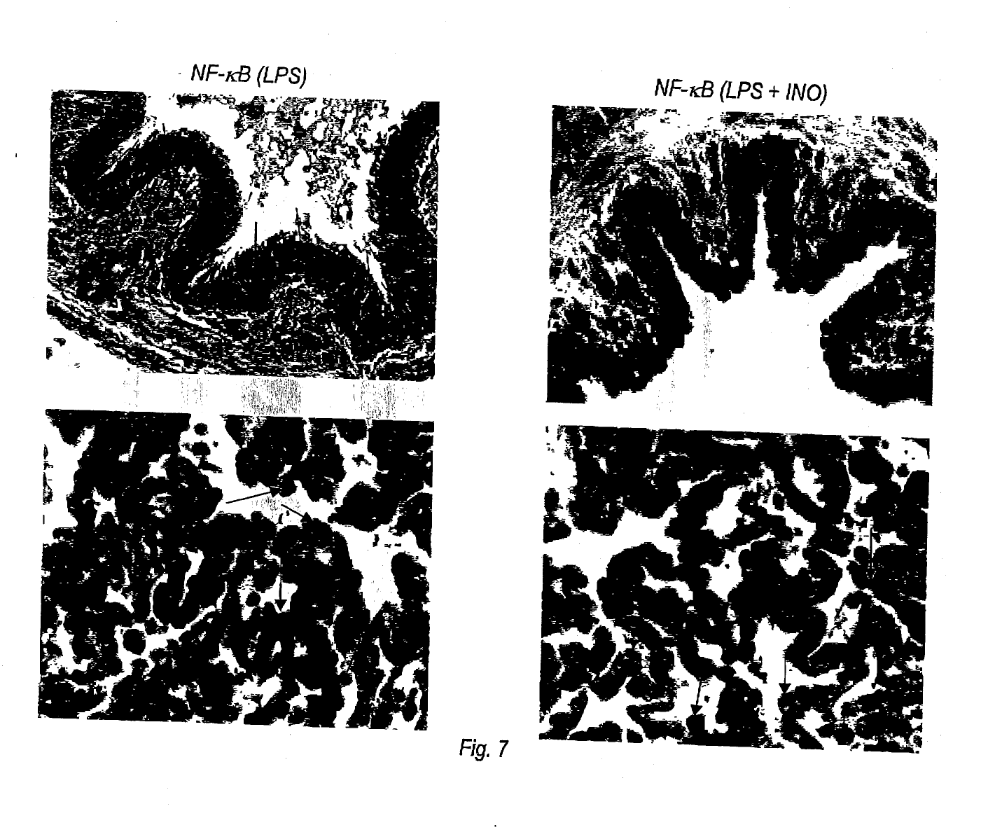

See Table 3 and FIG. 7.

FIG. 7 shows expression of NF-κB in lung tissue. Intense NF-kB expression (dark color) was seen in inflammatory cells, especially macrophages, as well as in some epithelial cells in bronchial walls. The NF-kB expression was located in the nuclei of the cells, and NF-κB positive cells had a tendency to aggregate. In the LPS+INO Group, NF-κB expression was at a low level, mostly located in cytoplasm and positive cells were sparse and scattered. Arrows indicate positively stained cells.

Table 3 shows expression of NF-κB in lung, liver and kidney tissue in piglets exposed to endotoxin alone (LPS), endotoxin+inhaled nitric oxide (LPS+INO), endotoxin+steroid (LPS+S), and endotoxin+inhaled nitric oxide and steroid (LPS+INO+S), compared with lungs from healthy piglets (Healthy). +indicates amount of expression. * means p<0.05 versus LPS. All types of changes were also significantly different from healthy controls, except for LPS+INO+S pigs that showed essentially normal expression in lung and liver.

TABLE 3
Expression of NF-kB (results of
immunohistochemical staining)

| Tissue | Healthy | LPS | LPS + INO | LPS + S | LPS + INO + S |
|---|---|---|---|---|---|
| Lung | + | ++++ | +(+)* | +++ | +* |
| Liver | + | ++++ | ++ | ++(+)* | +(+)* |
| Kidney | + | ++++ | ++(+) | +++ | ++* |

### DISCUSSION

This study has shown that endotoxin infusion in a pig model causes a severe inflammatory response, both in the pulmonary circulation (the lungs) and in the systemic circulation (liver and kidney). Moreover, endotoxin infusion caused almost complete elimination of the glucocorticoid receptor expression in lung tissue and liver (the kidney has not been studied so far with respect to GR) and an up-regulation of the inflammatory marker NF-κB. The inflammatory response consisted of edema, formation of thrombi, bleeding and ruptures of finer structures as e.g. glomeruli in the kidney and degeneration of lobuli in the liver. Commencement of inhaled NO therapy (INO) attenuated the inflammatory response but only to a limited degree. Another treatment modality, intravenous steroid administration, had almost no effect on the inflammatory response during the study period. Finally, the combination of INO and steroids had a remarkable effect in attenuating the inflammatory response. Some morphological abnormalities could still be seen, but tissue from the lung, kidney and liver were relatively similar to normal control tissue (FIG. 3-5).

Another observation was that physiological variables, mean pulmonary artery pressure and arterial oxygenation, were both almost normalized by the combined INO and steroid therapy. INO alone in the endotoxin model improved oxygenation and lowered MPAP but not back to normal and steroid therapy had almost no effect.

It is likely that the rather striking results that we have obtained can be explained in the following way. The endotoxin sepsis model triggers an inflammatory response with more or less complete down-regulation of the glucocorticoid receptor. Activation of this receptor prevents an inflammatory cascade response, and an early step in this cascade is release of NF-κB and other inflammatory markers. With a down-regulation of the GR the production and release of inflammatory markers is enhanced, promoting the sepsis process. (Molijn, G. J. et al., J. Clin. Endocrinol. Metab. (1995) 80(6), 1799-1803)

INO has a certain anti-inflammatory effect as well as endogenously produced NO via activation of inducible NO synthase (iNOS). A most important effect of INO, that we show for the first time, is the up-regulation of the GR expression in this septic condition. We assume that this has a key role for the regulation of the inflammatory process. We also assumed that an increased availability of the GR would enable a more efficient effect of a concomitant steroid therapy. With more glucocorticoid receptors available, an exogenous steroid administration may have more receptors to bind to and by these means more efficiently block the inflammatory process. Our findings support this assumption. (Kang, J. L. et al., J. Appl. Physiol. (2002) 92(2), 795-801; Kinsella, J. P. et al., Pediatr. Res. (1997) 41(4), 457-463; Webster, J. C. et al., Proc. Natl. Acad. Sci. USA (2001) 98(12), 6865-70; Almawi, W. Y. et al., J. Mol. Endocrinol. (2002) 28(2), 69-78; Smith, J. B. et al., Am. J. Physiol. Lung Cell Mol. Physiol. (2002) 283(3), L636-L647)

### CONCLUSION

In an endotoxin pig model the combined administration of INO and intravenous steroids markedly improved the histological appearance both in pulmonary and systemic organs (liver and kidney) and prevented edema formation, thrombus formation and structural damage. It is likely that this beneficial effect follows upon an up-regulation of the glucocorticoid receptor by INO, making steroid therapy more efficient.

1. A method of treating infectious inflammation in a mammal, including man, which comprises administering to a mammal in need of such treatment, nitric oxide, in the form of gaseous nitric oxide or a nitric oxide donor, in combination with a glucocorticoid, said combination being used in a therapeutically effective amount to accomplish treatment of said inflammation.

2. A method according to claim 1, wherein said infectious inflammation is caused by bacteria, fungi, viruses, mycoplasma, protozoa, helminths or insects.

3. A method according to claim 2, wherein said infectious inflammation is caused by bacteria.

4. A method according to any one of the preceding claims, wherein said infectious inflammation is selected from sinusitis, upper respiratory infection, bronchiectasis, bronchitis and chronic bronchitis, bacterial pneumonia, urinary tract infection, meningitis, acute respiratory distress syndrome, myocarditis, pericarditis, endocarditis, rheumatic fever, sepsis and septic arthritis.

5. A method according to claim 4, wherein said infectious inflammation is sepsis.

6. A method according to claim 4, wherein said infectious inflammation is bacterial pneumonia.

7. A method according to claim 4, wherein said infectious inflammation is acute respiratory distress syndrome.

8. A method according to any one of the preceding claims, wherein said medicament has a systemic effect.

9. A method according to any one of the preceding claims, wherein said medicament is in the form of a composition comprising said nitric oxide and said glucocorticoid for simultaneous administration thereof.

10. A method according to any one of claims 1 to 8, wherein said administering is a sequential administration of said nitric oxide and said glucocorticoid in any order.

11. A method according to claim 10, wherein said administering is a sequential administration of said nitric oxide and said glucocorticoid in said order.

12. A method according to any one of the preceding claims, wherein said gaseous nitric oxide is administered as inhalable nitric oxide.

13. A method according to any one of the preceding claims, wherein said glucocorticoid is administered intravenously.

14. A method according to claim 12, wherein said medicament is an inhalable medicament.

15. A method according to claim 12, wherein the concentration of gaseous nitric oxide to be inhaled is within the range of 0.1-180 ppm, preferably 1-80 ppm, and more preferably 1-40 ppm, said gaseous nitric oxide being present in a carrier gas or gas mixture.

16. A method according to any one of the preceding claims, wherein the dose of glucocorticoid is within the range of 0.1 to 10 mg/kg body weight.

17. A method according to any one of the preceding claims, wherein said nitric oxide donor is selected from S-nitrosocysteine, nitroprusside, nitrosoguanidine, isoamyInitrite, inorganic nitrite, azide, hydroxylamine, nitroglycerin, isosorbide dinitrate and pentaerithrityl tetranitrate.

18. A method according to any one of the preceding claims, wherein said glucocorticoid is selected from hydrocortisone, cortisone, corticosterone, prednisolone, prednisone, methylprednisolone, triamcinolone, dexamethasone, bethametasone, beclomethasone, budesonide, deoxycortone, fluocinoide, clobetasone and corticotrophin.

19. Pharmaceutical composition for treatment of infectious inflammation in a mammal, including man, which comprises nitric oxide, in the form of gaseous nitric oxide or a nitric oxide donor, in combination with a glucocorticoid, said nitric oxide and said glucocorticoid being present in a therapeutically affective amount to accomplish treatment of said inflammation.

20. Pharmaceutical composition according to claim 19, for use as defined in any one of claims 2 to 18.
